(19) United States
(12) Patent Application Publication
(10) Pub. No.: US 2005/0015064 A1
(43) Pub. Date: Jan. 20, 2005

(54) Skin cell perfusion unit

(76) Inventor: Joerg C. Gerlach, Pittsburgh, PA (US)

Correspondence Address: Joerg C. Gerlach MD, PhD, 3613 Butler St., Pittsburgh, PA, 15201, US

(21) Appl. No.: 10/866,294

(22) Filed: Jun. 12, 2004

(30) Foreign Application Priority Data
Jun. 13, 2003 (DE) 103 26 747.6

(51) Int. Cl. A61M 35/00; C12N 5/02

(52) U.S. Cl. 604/289; 604/290; 435/1.2; 435/297.1; 435/400; 435/401

(57) ABSTRACT

This invention concerns a unit for skin cell perfusion for the culture-, preservation-, proliferation-, and utilization of skin cells, skin fibroblasts, skin progenitor cells in vitro, as well as their maintenance in a skin wound of a patient. These perfusion units are constructed as one chamber that contains at least one membrane system to perfuse culture media or gases through the membrane. The membrane systems consist of flat and/or hollow fiber membranes whose outer surfaces may allow the cells to immobilize. The invention also concerns a perfusion station into which such perfusion units can be integrated as well as a method for cell culture, preservation, proliferation and utilization of such cells. Likewise, a method is described that supports the process of tissue engineering in a patient's wound, whereby the following functions are applied during the healing process: nutrition, oxygenation, detoxification, pH- and electrolyte balance, and dialysis. Perfusion units, like these, are applicable for regenerative cell transplantation associated with large skin wounds and severe burns, as well as to support the healing process of skin wounds.

This invention concerns a unit for skin cell perfusion for the culture-, preservation-, proliferation-, and utilization of skin cells, skin fibroblasts, skin progenitor cells in vitro, as well as their maintenance in a skin wound of a patient. These perfusion units are constructed as one chamber that is equipped with at least one port to inoculate the cells. In addition, the chamber contains at least one membrane system to perfuse culture media or gases through the membrane whereby the membrane system is equipped with at least one supply- and removal line. The membrane systems consist of flat and/or hollow fiber membranes whose outer surfaces may allow the cells to immobilize. The invention also concerns a perfusion station into which such perfusion units can be integrated as well as a method for cell culture, preservation, proliferation and utilization of such cells. Likewise, a method is described that supports the process of tissue engineering in a patient's wound, whereby the following functions are applied during the healing process: nutrition, oxygenation, detoxification, pH- and electrolyte balance, and dialysis. Perfusion units, like these, are applicable for regenerative cell transplantation associated with large skin wounds and severe burns, as well as to support the healing process of skin wounds.

While injuries, such as skin burns to the most upper layer of the skin (epidermis, keratinocytes) usually heal by themselves, larger wounds that reach the regeneration layer of the skin (basal keratinocytes, skin progenitor cells) are not able to do that.

Such injuries require the so-called split-skin-technique where the skin, that is to be transplanted, is taken from a healthy area and fanned out into many small sections. In cases of large area burns this classic form of transplantation is however limited due to the lack of ample availability of healthy skin areas; particularly in cases of burn injuries the loss of adult stem cells is insurmountable.

The culture of keratinocyte cells is well established. Devices to culture skin cells, like Petri dishes, flasks, or culture bags, are well known.

First clinical results of the transplantation of keratocytes cultured in flasks are described in: Carsin H, Ainaud P, Le Bever H, Rives J M, Le Coadou A, Stephanazzi J. Objectives, results and future prospects of burn treatment in 1997. Bull Acad Natl Med 1997 October; 181 (7):1307-19; discussion 1319-20 und Phillips T J, Gilchrest B A,: Clinical application of cultured epithelium. Epith Cell Biol 1992; 1:39-46 und Still J M, Orlet H K, Law E J. Use of cultured epidermal autografts in the treatment of large burns. Burns 1994; 20:539-541.

Skin cells were isolated from small skin samples that were taken from a patient. These samples proliferated in Petri dishes and were placed on the patient's wound as regenerated cell transplants. The bio technological service to generate skin cell transplants from the patient's own skin source is offered by various organizations, for example Genzyme, Boston, USA.

These established techniques, however, exhibit poor cell supply during the attachment phase. This results in water-, electrolyte, and pH displacement as well as toxin built-up. This non-physiological biomatrix in the wound prohibits an optimal attachment and proliferation of the transplanted cells. The application of two-dimensional closed cell layers encourages the development of blisters underneath those layers, which results in the detachment of the transplanted cells from their blood supply. In cases of burn injuries the loss of protein as result of dehydration as well as the accumulation of toxins in the wound have to be addressed.

Addressing those problems, the invention reduces the loss of heat, proteins, and fluids from the wound during the first clinical phase and at the same time ensure a germ protective wound dressing, as well as reduce electrolyte/pH displacements, and the accumulation of toxins. Thereby, this perfusion unit improves the results of treating skin defects with skin cells in a later clinical phase.

Afore mentioned problems are solved with the skin-cell-perfusion-unit by way of the characteristics according to claim 1, with the perfusion station by way of the characteristics according to claim 12, and through the procedure by way of the characteristics according to claim 17. Claim 21 describes the application of the perfusion unit. The other dependent claims point out advantageous further advancements.

The perfusion unit for the culture-, preservation-, proliferation-, and utilization of skin cells, skin fibroblasts and/or progenitor cells provides at least one chamber with at least one canal for cell supply. The inside of the chamber contains a membrane system consisting of at least one hollow fiber or flat sheet membrane whose outer surface may allow for the immobilization of cells. The membrane is connected with at least one supply line and at least one removal line to allow the perfusion of culture media and/or gases through the membrane. Furthermore, it is important that at least one surface of the chamber is constructed as contact area that can be arranged on skin wound surfaces and has a reversible, and germ tight removable cover, e.g. a foil.

Equally important is the high flexibility of the membrane system to be able to adjust to different body shapes in problem areas like joints.

The perfusion unit serves for in vitro cultivation of, preferably, the patient's own keratinocytes and/or adult stem cells of the skin (progenitor cells), and cells of the skin's connective tissue (fibroblasts). The in vitro culture phases is developed in such a way that the cells spread out on, or aggregate between, the membranes in island like groupings, and not grow together in confluence. Likewise, co-culture of different kinds of cells can be performed side by side in island like groupings, so that during the in vitro phase a closed two-dimensional new skin layer formation is prevented.

In a later phase the perfusion unit simplifies the transfer of those cells into the wound. These units with the transferred cells, which are subsequently supplied with nourishment through the membrane with culture media and oxygen, support the process of tissue engineering in the wound. The membrane supports this process of tissue engineering during a phase in which the transferred cells are not yet supported by the patient's own vascular system. This kind of wound treatment allows for the warming, pH- and electrolyte regulation, detoxification, and reduction of fluid loss of the wound in a similar way as via dialysis membranes.

Preferably, the membrane inside the chamber is built from several hollow fibers arranged in parallel. All hollow fibers are arranged in such a way that each of the ends is connected to the input and output lines.

An alternative construction of the interior chamber is that at least one hollow fiber inside the chamber is arranged in a meandering way. This means that the input line is connected to one side of the hollow fiber and the output line to the other end of the fiber, which allows free transportation of culture media and/or gases through the hollow fiber. Preferably, the perfusion unit can be constructed with several chambers. This allows for a serial connection of several chambers, which permits continuous, successive flow of culture media and/or gases through the individual chambers. Alternatively it is also possible to arrange several chambers in parallel order.

Furthermore it is suggested to use chambers and/or membranes that, after being applied to the patient's wound and successful cell growth, don't have to be removed mechanically causing anew injury. This is possible in case the chambers and/or membranes consist of a material that can be reabsorbed and/or digested by the patient's tissue.

Another preferred arrangement is that the chamber and/or membranes consist of a material that can be dissolved by enzymes. Particularly applicable are materials like cellulose and/or cellulose containing compounds. These materials are digestible by the enzyme cellulase. Because cellulase is a plant derivative it does not attack human cells. Simultaneously, cellulase is biocompatible, harmless to skin cells, and applicable for external patient use.

Furthermore, materials like collagen are preferred because they are digestible by collagenase, or even the patient's own tissue collagenase.

Preferably, the perfusion unit has a flexible surface area that is adjustable to the body's contours. This permits excellent application possibilities of the perfusion unit on areas of the body difficult to access, like joints. Likewise, the membrane inside the perfusion unit can be designed flexible. The membrane system can also be removable from the perfusion unit while the connections to the perfusion station remain. Then, afore mentioned functions in the wound are warranted exclusively via the membrane system after the membrane is placed on the wound with an appropriate wound cover.

A perfusion station is provided by the invention that contains at least one perfusion unit as described afore. The perfusion unit is connected with at least one supply-and drainage reservoir via respective inlets and outlets. The supply reservoir contains culture media and/or gases, necessary for the perfusion through the membrane, which will be collected to the drainage reservoir after passing through the membrane.

Preferably the perfusion media is tempered to body temperature and oxygenated by the device.

It is also possible that a connection exists between the supply- and drainage reservoir to constantly flush the membrane with culture media and/or gases in a circular flow.

Another advantageous feature exhibits connectors, such as Luer-Locks and/or clamps between the inlet and the supply reservoirs and the unit.

Preferably the perfusion station is equipped with a pump that pumps culture media and/or gases through the membrane. Thereby it is preferred to maintain an exact perfusion rate and hydrostatic pressure in the wound through a pressure regulator. Alternatively, perfusion is also possible hydraulically rather than through a pump. Therefore, the supply- and output reservoir levels relative to the patient have to be arranged in such a way that hydraulic transportation of culture medium and/or gases can occur. Here, the levels can adjust the pressure in the membrane.

A method for the culture, preservation, proliferation, and utilization of skin cells, fibroblasts and/or progenitor cells using afore described perfusion unit is provided. The procedure is based on the fact that, in vitro, e.g. in afore described perfusion unit, the chamber is perfused with culture media and/or gas to facilitate the growth of immobilized skin cells on the membrane surface. First the cells are brought into the device via a supply port. Then the device will be swiveled several times in all directions in intervals of 1-60 minutes. During this time perfusion continues until island like groups of skin cells have developed on the membrane surface.

In contrast it is less desirable to see cohesive layers of skin cells developing on the membrane surface or in the unit.

The infusion of cells into the device preferably occurs through individual cells with a dilution rate that will lead to cell growth of individual cells, namely groups of cells with low density. Subsequent cell growth will lead to larger, individual, and sporadic growth of cell islets. Therewith, if necessary for the cell-passage, trypsinization and removal is simplified. Seeding cells leading to islets also reduces the need to trypsinize because the cells have more room to grow. Later transfer of cells from the membranes in the wound will also improve by using individual groups of cells.

The application of this invention has advantageous compared to the commonly used Petri dish based methods.
Foregoing confluent growth that is associated with reduced cell cycles, results in a reduced time need for the phase of cell proliferation in vitro.
The system allows for automated cell treatment/development under the condition of a closed system, avoiding the risk of human error. Therefore, automated trypsinization in a closed system is possible.
Overall the system yields a reduction in processing costs.
The system is developed in such a way that enlargement and reduction is easily scalable, and particularly the customization to different sizes of various body parts is possible.
The system allows insular cell cultures with co-cultures of various cells insularly next to each other.

Such generated insular skin cell groups can subsequently be placed with the membranes on a skin wound, where the perfusion process of the membrane with culture media and/or gas can proceed and cell supply growth can continue.

The perfusion unit is particularly useful in medicine for the purpose of wound coverage and wound treatment. Especially for the treatment of burn patients, the perfusion unit is useful for the provision of cells. It is also advantageous for the supply of the transferred cells supporting tissue engineering in the wound.

The perfusion unit is particularly preferred for the application of cell cultures from skin cells, fibroblasts and/or progenitor cells onto skin wounds. This includes the application of insular cell groups from the respective (autologous) cells.

The system allows monitoring the cells via microscopy, e.g. time-laps video microscopy whereby the cells remain visible edgewise on the capillaries.

The perfusion unit also permits in vitro culture of cells for research purposes in the sense of an analytical bioreactor by using transparent materials.

A typical sequence of the application in five steps is described as follows:

Step One:

Hydrolization of the membrane (if required) in the perfusion unit, if necessary with autologous patient plasma. Alternatively with a coating of biomatrix, e.g. fibrin, which can, for instance, be applied as fibrin adhesive in spray form.

Step Two:

Seeding and culturing of cells in the perfusion unit that serves as the culture system for the cell proliferation.

Step Three:

During the cell transport from he laboratory to the patient the perfusion unit serves as a safe and sterile container in which the cells continue to remain in culture condition.

Step Four:

After opening the perfusion unit, the membrane system, which previously was an integral part of the bioreactor, serves to transfer cells into the wound. The membrane itself only has to remain in the wound intermittently until the cells are well adhered in the wound.

Step Five:

After the fourth step, the membrane system based on its continuing ability to be perfused, preferably serves to improve the cell adhesion during the first phase of the first and second day, -, temperature-, and water regulation as well as detoxification in pH the sense of dialysis.

The perfusion unit can also be used for in-vitro reconstruction of a multi layered, organ-typical skin construction. Like wise, it facilitates in-vivo reconstruction of the skin configuration in the patient's wound.

The skin cell perfusion unit can also be applied to the patient's wound by independently inserting the previously, separately, cultivated cells into the wound immediately before applying the perfusion unit. Based on its ability to perfuse the membranes with oxygen and nutrients and the resulting dialysis effect (water-, ph-, electrolyte balance, detoxification, warming, etc.), during the first phase of the first two days the membrane system serves to improve the adhesion of the cells and tissue engineering in the wound.

The unit can also serve as in vitro skin cell bioreactor for research and/or commercial use. The following figures shall illustrate the device to be registered without restricting it to its design.

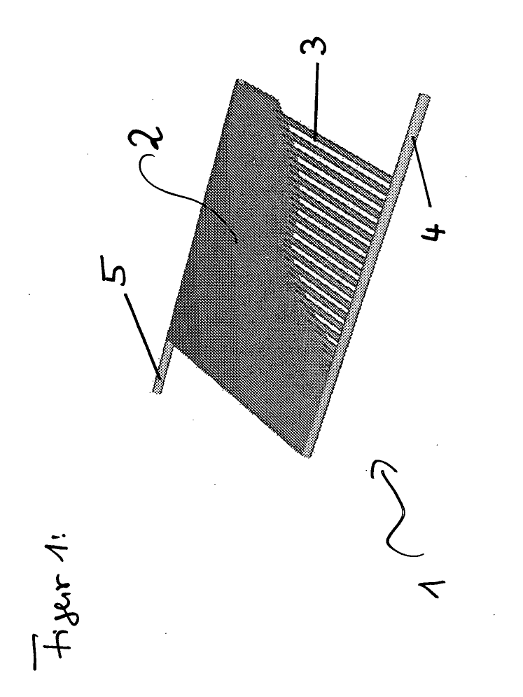

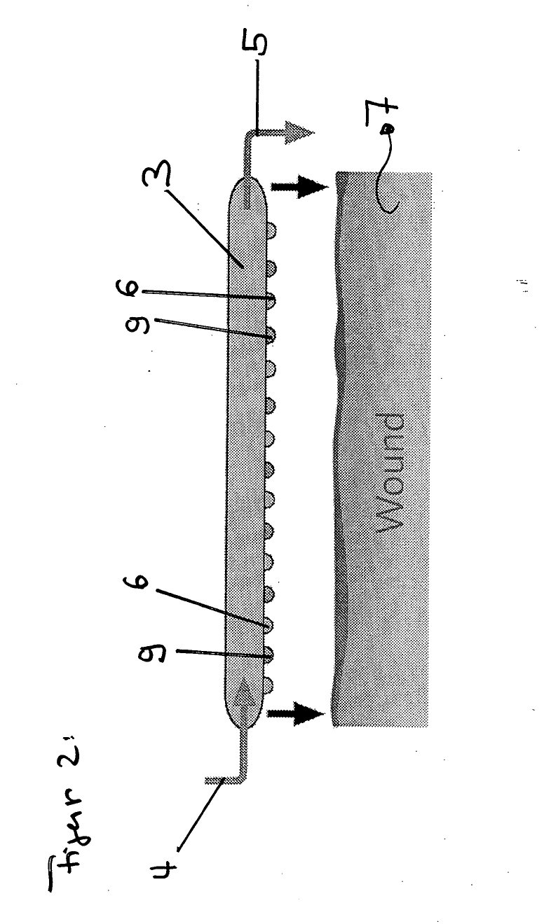

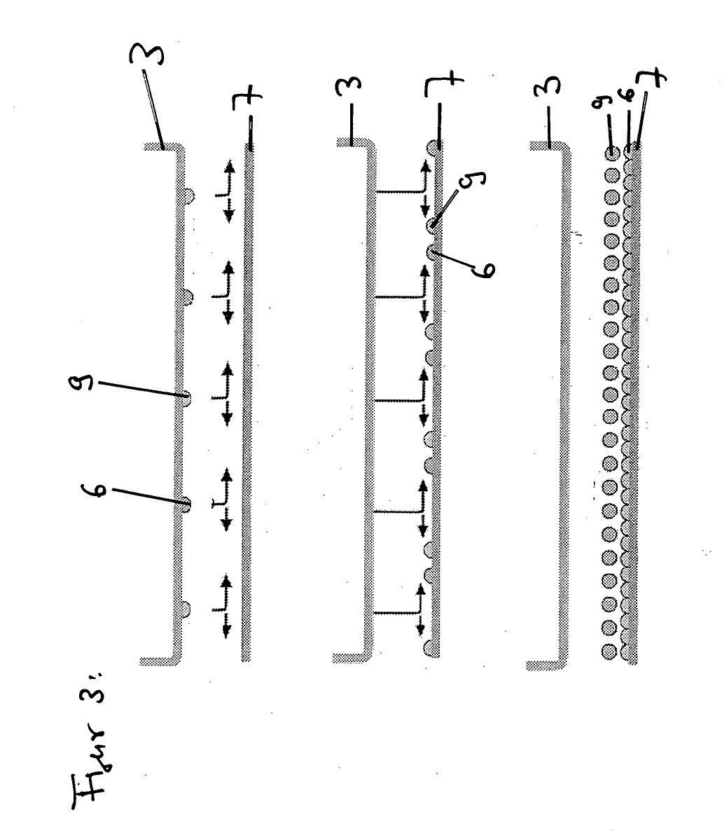

FIG. 1 describes a schematic illustration of the structure of a perfusion unit FIG. 2 schematically describes the process of bringing a perfusion unit in contact with a wound FIG. 3 schematically describes the process of cell culture and proliferation of the described skin cells in a wound

FIG. 1 shows the schematic design of a perfusion unit 1. The perfusion unit is composed of a chamber with two exterior walls 2 between which a number of hollow fibers 3 are integrated. At one end the hollow fibers are connected to an input line 4 and on the other end the hollow fibers 3 are connected to an output line 5. Now culture media and/or gas, e.g. oxygen, can be transported through the input line 4, flush the hollow fibers 3, and be removed through the output line 5. The exterior chamber wall 2 described in FIG. 1 is simply realized as two affixed foils above and below the membrane. The chamber is created by affixation and fusion of the two foils including the lines 4 and 5. This figure does not depict the port for the cell infusion into the chamber. This port can be affixed at arbitrary positions on the chamber to enable the transportation of the cells into the space between the chamber walls and hollow fibers where they can become immobilized. It is also possible that, inside the chamber, two or more membranes are arranged on top of each other to allow metabolic exchange in counter current process. The exterior walls of the chamber 2 can also be developed to be permeable for oxygen and carbon monoxide. This also does not depict possible further housing elements, which allow easy use in vitro and provide sterile layers for the final sterile removal of the membrane systems before placing into a wound.

FIG. 2 describes how the membrane of the perfusion unit is brought into contact with a wound. It schematically demonstrates a hollow fiber 3 at which skin fibroblasts 6 and skin progenitor cells 9 are immobilized on the side facing the wound. Simultaneously the perfusion of culture media and/or gases through the hollow fiber 3 occurs via the input line. Subsequently the culture media and/or gases can be removed via output line 5. The hollow fiber prepared in such a way can now be placed on the wound to facilitate subsequent skin cell culture in the wound 7, namely tissue engineering. Furthermore, the cells can also be separately placed in the wound beforehand.

FIG. 3 describes the process of in-vivo reconstruction of skin construction in the wound. FIG. 3a) depicts the attachment of skin cells into the wound 7, consisting of skin progenitor cells 9 and skin fibroblasts 6 that are insularly immobilized on the side of the hollow fiber facing the wound surface. FIG. 3b) describes the subsequent growth of skin cells in the wound. FIG. 3c) describes the following process of a closed development of skin layers 10 in the wound 7. For this purpose it is necessary to continue the perfusion with culture media and/or gases through the hollow fiber, which, by itself, guarantees sufficient supply of the cells and cell growth.

1. Perfusion unit for wound treatment, cell culture-, preservation-, proliferation-, transfer-, and utilization of skin cells, skin fibroblasts and/or progenitor cells consisting of at least one chamber with at least one port for cell supply, at least one membrane connected to at least one input line and at least one output line arranged inside the chamber for the perfusion of culture media and/or gases through the membrane. The membrane system consists of at least one hollow fiber on which the cells can immobilize, whereby at least one surface of the chamber is developed as contact area on a patient's skin wound surface and exhibits a reversible, sterile and removable cover.

2. Perfusion unit according to claim 1, thereby characterized that the perfusion unit exhibits a flexible contact area that is adaptable to the body's contours.

3. Perfusion unit according to claim 1 or 2, thereby characterized that the membrane inside the chamber consists of several hollow fibers arranged in parallel.

4. Perfusion unit according to claim 1 or 2, thereby characterized that at least one hollow fiber inside the chamber is arranged in a meandering way.

5. Perfusion unit according to one of afore mentioned claims, thereby characterized that at least two membrane systems inside the chamber (according to claim 1-4) are arranged on top of each other or alternating, which are perfuseable in counter current flow, and the exterior wall of the chamber consists of a flat membrane permeable to oxygen.

6. Perfusion unit according to one of afore mentioned claims, thereby characterized that the chamber consists of a flat membrane permeable for oxygen.

7. Perfusion unit according to at least one of afore mentioned claims, thereby characterized that the chamber and/or membrane consist of a digestible and/or bioabsorbable material.

8. Perfusion unit according to at least one of afore mentioned claims, thereby characterized that the chamber and/or membrane consist of a material that is dissolvable by enzymes.

9. Perfusion unit according to at least one of afore mentioned claims, thereby characterized that the chamber and/or membrane consist of cellulose and/or cellulose containing compounds.

10. Perfusion unit according to at least one of afore mentioned claims, thereby characterized that the membrane consists of collagen.

11. Perfusion unit according to at least one of afore mentioned claims, thereby characterized that several chambers are connected with each other in series.

12. Perfusion unit according to at least one afore mentioned claims, thereby characterized that several chambers are arranged parallel.

13. Perfusion station containing at least one perfusion unit according to at least one of the claims 1-11, at least one inlet is connected to a supply reservoir containing culture medium and/or gases, and at least one outlet connected to an outflow reservoir, which collects the perfusion medium and/or gases that perfuse the membrane.

14. Perfusion station according to claim 12, thereby characterized that here is at least one clamp between the input line and the supply reservoir and/or between the output line and the run off reservoir.

15. Perfusion station according to at least one claim 12 or 13, thereby characterized that the perfusion station exhibits a pump station with pressure control for the perfusion of culture media and/or gases through the membrane.

16. Perfusion station according to at least one claim 12-14, thereby characterized that the supply reservoir and the run off reservoir are arranged in such a way that the perfusion and/or pressure adjustment can occur hydraulically.

17. Perfusion station according to at least one claim 12-15, thereby characterized that it exhibits a heater and/or oxygenator to heat and/or oxygenate the culture media.

18. Processes for cell culture-, preservation-, proliferation-, transfer-, and utilization of skin cells, fibroblasts and/or progenitor cells under the application of the perfusion unit according to at least one of the claims 1-12 where the perfusion unit consists of at least on chamber; whereby the chamber is perfused with culture media and/or gas to sustain the growth of the cells, which are immobilizes on the membrane surface, until the development of insular cell areas on the membrane surface.

19. Process according to claim 17, thereby characterized that the perfusion unit is placed on a skin surface while the perfusion with culture media and/or gas continues.

20. Process according to claim 17 or 18, thereby characterized that the perfusion unit consists of several chambers with insular cell areas that are arranged side by side, whereby keratinocytes, skin fibroblasts and/or progenitor cells are cultivated side by side, separately, and insularly.

21. Process according to claim 17 to 19, thereby characterized that subsequently the perfusion unit will undergo enzymatic digestion and/or decomposition, e.g. with collagenase or cellulase.

22. Utilization of the perfusion unit according to at least one of the claims 1-11 for wound coverage in medicine

23. Utilization of the perfusion unit according to at least one of the claims 1-11 for the application of cell cultures from skin cells, skin fibroblasts and/or progenitor cells to skin wounds

24. Utilization of the perfusion unit according to at least one of the claims 1-11 for the application of cell islets from autologous skin cells, skin fibroblasts and/or progenitor cells to a skin surface.

25. Utilization of the perfusion unit according to at least one of the claims 1-11 for in vitro reconstruction of a multi layered, organ typical skin construction that follows the physiological skin construction.

26. same as 24.

27. Utilization of the perfusion unit according to at least one of the claims 1-11 to support tissue engineering in the wound through temperature supply, oxygenation, nutrition, fluid- and/or pH- and electrolyte balance, detoxification, as well as dialysis in the wound.

28. Utilization of the perfusion unit according to at least one of the claims 1-11 to support tissue engineering in the wound after separate or simultaneous cell application in the wound.

29. Utilization of the perfusion unit according to at least one of the claims 1-11 to support tissue engineering in the wound during simultaneous application of progenitor cells from an autologous bone marrow biopsy.

30. Utilization of the perfusion unit according to at least one of the claims 1-11 for the application in a wound without additional cell import to support the healing process via temperature supply, oxygenation, nutrition, fluid- and/or pH- and electrolyte balance, detoxification, as well as dialysis in the wound.

31. Utilization of the perfusion unit according to at least one of the claims 1-11 for cell culture-, preservation-, differentiation-, proliferation-, transfer-, and/or utilization of individual cell types, and/or various cell types (co-cultures) of an organ.

32. Utilization according to claim 31 for stem cells

33. Utilization according to claim 31 for human cells

34. Utilization according to claim 31 for adult stem cells

35. Utilization according to claim 31 for the production of cells

36. Utilization according to claim 31 for the production of progenitor cells and cell transplantation

37. Utilization according to claim 31 for the production of gene technologically modified cells, immortal cells, and/or trans-genetic cells.

38. Utilization of the module according to one of the claims 1-29 for the production of substances via cells.

39. Utilization of the module according to one of the claims 1-29 for the differentiation of organ typical cells from stem cells.

40. Utilization of the module according to one of the claims 1-29 to generate organ typical structures from adult stem cells, bone marrow or embryonic cells.

41. Utilization of the module according to one of the claims 1-29 as extracorporeal hybrid organ for organ support, e.g. extracorporeal skin cell perfusion for skin regeneration with the help of skin cell growth factor production.

42. Utilization of the module according to one of the claims 1-29 as implantable organ transplant.

43. Utilization of the module according to one of the claims 1-29 for test- and analytical purposes and as laboratory system to replace and/or supplement animal research.

44. Utilization of the module according to one of the claims 1-29 as in vitro virus culture and virus reproduction.

45. Utilization of the module according to one of the claims 1-29 as a system to manufacture vaccines.

46. Utilization of the module according to one of the claims 1-32 as in vitro placenta model and cell growth in placenta tissue.
